(19) United States
(12) Patent Application Publication
(10) Pub. No.: US 2015/0327894 A1
(43) Pub. Date: Nov. 19, 2015

(54) PLATE CENTERING DEVICE FOR ORTHOPEDIC SURGERY

(71) Applicants: Mauricio TORO, (US); Jairo Fernando GOMEZ, (US); INDUSTRIAS MEDICAS SAMPEDRO S.A.S., Sabaneta (CO)

(72) Inventors: Mauricio TORO, Sabaneta (CO); Jairo Fernando GOMEZ, Sabaneta (CO)

(21) Appl. No.: 14/652,812

(22) PCT Filed: Dec. 19, 2012
(86) PCT No.: PCT/IB2012/057493

§ 371 Date: Jun. 17, 2015

(51) Int. Cl. A61B 17/80; A61B 17/88

(52) U.S. Cl. CPC A61B 17/808; A61B 17/8872; A61B 17/8061

(57) ABSTRACT

The present invention relates to a device used for centering a plate during orthopedic surgery, especially of the distal radius, where said plate centering device is manufactured from polymeric, highly elastic material that allows it to adapt to different bone morphologies. Thus, the plate centering device is used in orthopedic surgery, particularly in those related to peri-articular fractures, in which the commonly used practice is to attach the plate starting at the epiphysiary zone, where it is common to lose the plate's orientation with the remaining bone hence difficulting the fracture's reduction, making the surgical process more complicated. The plate centering device, by being elastic, wraps around the bone at the diaphysary portion, guaranteeing that the plate does not lose its orientation while it is being attached to the epiphisary portion. This attachment can be supported, if necessary, by a clamp that increases the pressure on the bone, such as the clamps normally used by surgeons in the operating room when carrying out this type of surgery.

### CROSS-REFERENCE TO RELATED APPLICATION

This application claims priority to PCT Application No. PCT/IB2012/057493 having a filing date of Dec. 19, 2012, the entire content of which is hereby incorporated by reference.

### FIELD OF TECHNOLOGY

The following relates to a device used for centering a plate during orthopedic surgery, especially of the distal radius, where said plate centering device is manufactured from polymeric, highly elastic material that allows it to adapt to different bone morphologies. Thus, the plate centering device is used in orthopedic surgery, particularly in those related to peri-articular fractures, in which the commonly used practice is to attach the plate starting at the epiphysiary zone. Further, it is common when attaching this zone to lose the plate's orientation with the remaining bone hence making difficult the fracture's reduction, making the surgical process more complicated.

In this manner, the plate centering device of embodiments of the present invention, by being elastic it wraps around the bone at the diaphysary portion, guaranteeing that the plate does not lose its orientation while it is being attached to the epiphisary portion. This attachment can be supported, if necessary, by a clamp that increases the pressure on the bone, such as the clamps normally used by surgeons in the operating room when carrying out operation of this type, where said clamp allows for the centering device to be attached by means of a central channel covering all the outer portion of said centering device and allows both the surgeon as well as any of his assistant's during the surgery to keep the plate in place while the surgery is being carried out.

### BACKGROUND

Currently, most orthopedic surgeries related to fractures and requiring installation of a plate for joining the fractured bone, it is carried out by means of a complex procedure which also requires a longer recovery by the patient. For this reason, it is necessary for said procedure to be carried out in a successful manner and only once, in other words, taking into consideration that both the surgery and the recovery are traumatic for the patient, it is undesirable for there to be any inconvenient with the plates or the bonding of the broken bone, so the patient can have an adequate recovery.

In this way, the plates commonly used in these type of procures require a series of screws and accessories or additional elements that must keep them in place, preventing it from moving and producing an undesired bone regeneration which could lead to malformation on the patient's bones. For this it is necessary that the plate is placed on its implant location and attached, which is commonly carried out by means of a series of clamps that let the plate to be in place while the operation is carried out, but once it is over they are removed such that only the screws keep said plate in place, which is not always desirable in every case since there can be movements of the plate and an inadequate bone regeneration.

### SUMMARY

An aspect relates to useful devices when carrying out orthopedic surgery where it is required that the plate being installed on the patient must remain in its corresponding position and, in that way, avoid problems due to a bad placement of the plate during surgery.

In this sense, document CN 1686057 refers to an adaptation plate centering device, where said device is of the type with centered teeth for treating bone fractures, and said device is adequate for installing bone plates during surgery, where the same is comprised of an arc base plate with an arc cross section, screw holes, holes for installing pressure screws and bone regulator holes, hooked shaped angular side undulations (ribs) with a cutting tip, wherein the teeth are arranged on the cutting tip of the longitudinal angular rib at intervals, such that the device couples with the fracture site and does not move, just as it is necessary for this type of treatment or surgery.

Nonetheless, embodiments of the invention described in this anteriority presents a series of teeth on the inner part allowing the device to remain in position and not move, which fact could be regarded as a disadvantage since said teeth do not assure that the plate is kept in place, since also there is no device, such as an inner guide, so there is no displacement of the plate.

On the other hand, document EP 1943967 discloses a centering device for orthopedic surgery, which is used in operations requiring intra-medullary binding, where said device is comprised of a metallic frame of a “C” shaped semi-circular section provided with an amplifier pointed towards an X-ray tube, which comprises at least one fixed bar attached to the same C-shaped frame and wherein it also comprises a series of mobile means cooperating with the incident beam emitted by the X-ray tube.

Nonetheless, embodiments of the invention described in this document presents the disadvantage of requiring a series of devices for a surgery carried out by means of the use of X-ray and complex electronic systems, which is undesirable since it increases the costs associated with the procedure.

Now, document U.S. 2011238184 is related with methods and instruments for interbody fusion, which are useful when carrying out laparoscopic surgery for preparing a site for implanting a fusion device or implant. The device mentioned in this document allows to carry out the centering of a system for laparoscopic surgery by means of a device which is inserted in the patient's bone and allows the laparoscopy screw to pass through the place desired by the surgeon.

Nonetheless, the device described in this anteriority has the disadvantage of having to be implanted directly into the patient's bone and acts as a guide for the laparoscopy device, increasing at the same time the patient's recovery due to the implanted device.

According to the above, it can be clearly seen that there exists in the state of the art a need to design and implement a device to be used in orthopedic surgery, which allows to keep a bone coupling plate in its place and centering it when said procedure is being carried out, where it is required for the device to be easy to use, economic and also counts with a system to be used with the clamps commonly used by surgeons of these types of surgeries, wherein it is also desirable that the plate centering device is manufactured in a highly elastic polymeric material, that allows the same to adapt to different bone morphologies, taking into consideration that every patient's bones are different.

### BRIEF DESCRIPTION

Some of the embodiments will be described in detail, with reference to the following figures, wherein like designations denote like members, wherein:

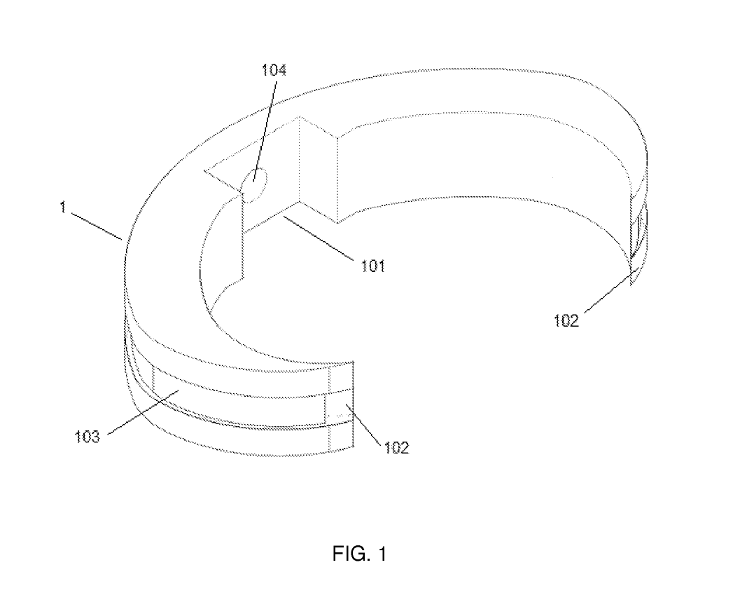

FIG. 1 corresponds to a perspective view of the plate centering device of embodiments of the present invention;

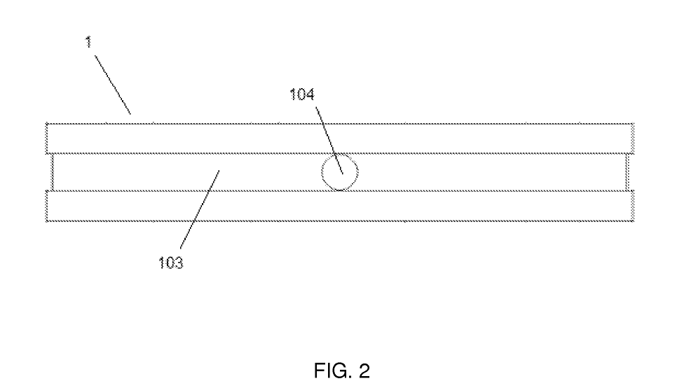

FIG. 2 corresponds to a top view of the centering device of FIG. 1;

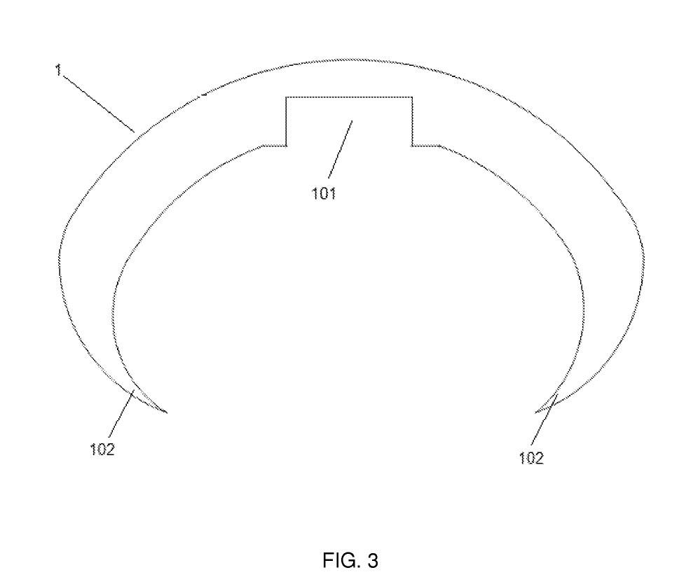

FIG. 3 corresponds to a front view of the centering device of FIG. 1;

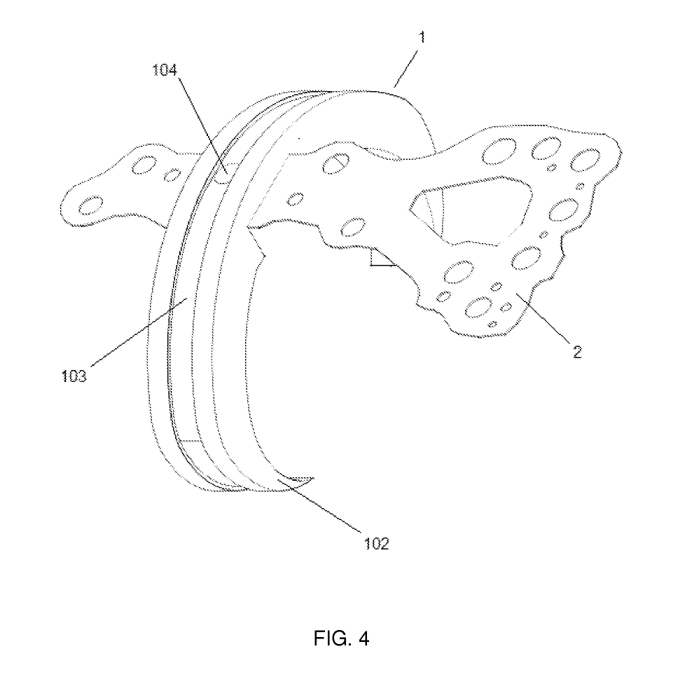

FIG. 4 corresponds to a perspective view of the device of FIG. 1 along with a plate for orthopedic surgery and

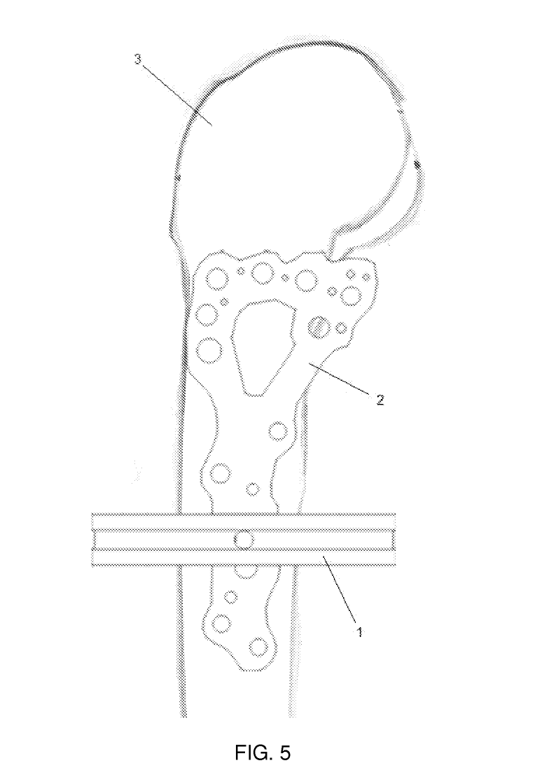

FIG. 5 corresponds to a top view of the set in FIG. 4 installed on a patient's bone.

### DETAILED DESCRIPTION

The centering device (1) for a plate (2) to be used in orthopedic surgery, preferably distal radius surgery, of embodiments of the present invention comprises the following components or parts:
A “C” shaped central body including a couple of ends (102);
A central channel (101) which is on the middle portion of the central body between both ends (102) and is used to place the plate (2) to be used during surgery;
A fastening channel (103) surrounding the outer edge of the centering device (1) and allowing said device (1) to be secured by the surgeon by means of clamps; and
A fastening orifice (104) located on the inside portion of the central channel (101) and allowing the device to be attached to the patient's bone (3) by means of a screw that goes through it along with the plate (2)

Therefore, the centering device (1) is used to center the plate (2) during orthopedic surgery, special distal radius surgery. In a similar way, the device (1) is used on peri-articular fracture surgery, where device (1) is placed around the bone (3) where the plate (2) is going to be installed to correct or repair the fracture, as shown in FIG. 5.

Similarly, the central channel (101) has a width corresponding exactly to one of the widths of plate (2), which depends directly on the type of surgery being carried out, in other words, depends on the fractured bone to be mended.

In an embodiment of the present invention, the ends (102) finish on a tip, in other words, the outer edge and the inner edge meet at a tip on each of both ends (102), so as to allow the device (1) to be fitted to the patient in an exact shape and preventing an excess material from the body of the centering device 81), thus reducing the manufacturing costs thereof

In a preferred embodiment, the centering device (1) is manufactured form a highly elastic polymeric material, which allows it to adapt to the different bone morphologies. In this manner, by being elastic the centering device (1) is wrapped around the bone on the diaphysary portion, guaranteeing that the plate 82) does not lose its orientation while it is being attached to the epiphysiary portion, wherein said attachment can be supported by a clamp that increases pressure on the bone, where the clamp grabs the device (1) by means of channel (103).

Although the present invention has been disclosed in the form of preferred embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

1. A centering device for a plate to be used in orthopedic surgery, characterized in that it comprises the following components or parts:
a “C” shaped central body including a couple of ends;
a central channel which is on the middle portion of the central body between both ends and is used to place the plate to be used during surgery;
a fastening channel surrounding the outer edge of the centering device and allowing said device to be secured by the surgeon by means of clamps; and
a fastening orifice located on the inside portion of the central channel and allowing the device to be attached to the patient's bone by means of a screw that goes through it along with the plate.

2. The centering device for a plate according to claim 1, wherein the central channel has a width corresponding exactly to one of the widths of plate (2), which depends directly on the type of surgery being carried out.

3. The centering device for a plate according to claim 1, wherein the ends finish on a tip, in other words, the outer edge and the inner edge meet at a tip on each of both ends.

4. The centering device for a plate according to claim 1, wherein the centering device is manufactured form a highly elastic polymeric material, which allows it to adapt to the different bone morphologies.
